(19) United States
(12) Patent Application Publication
(10) Pub. No.: US 2005/0058614 A1
(43) Pub. Date: Mar. 17, 2005

(54) Methods for the treatment of gray hair using cyclopentane(ene) heptan(en)oic acid amides

(75) Inventor: Achim H. Krauss, Foothill Ranch, CA (US)

Correspondence Address: Robert J. Baran (T2-7H);ALLERGAN, INC., Legal Department, 2525 Dupont Drive, Irvine, CA, 92612, US

(73) Assignee: Allergan, Inc.

(21) Appl. No.: 10/663,014

(22) Filed: Sep. 15, 2003

(51) Int. Cl. A61K 7/06; A61K 31/557

(52) U.S. Cl. 424/70.1; 514/573

(57) ABSTRACT

The present invention relates to novel pharmaceutical compositions of bimatoprost and other cyclopentane(ene) heptan(en)oic acid amides and their use in preventing gray hair or promoting the conversion of gray hair to the original pigment in hair follicles.

### FIELD OF THE INVENTION

The present invention relates to the use of bimatoprost and other cyclopentane(ene) heptan(en)oic acid amides for the prevention of gray hair in mammals, e.g. humans or the conversion of gray hair of humans or other mammalian hair to the original pigment in mammal, e.g. human, hair follicles.

### BACKGROUND OF THE INVENTION

U.S. patent application Ser. No. 10/345,788, which is hereby incorporated by reference, discloses the use of bimatoprost for enhancing hair growth. Bimatoprost, which is sold by Allergan, Inc. of Irvine, Calif., U.S.A. as Lumigan® ophthalmic solution, for treating glaucoma now has been found as being effective to increase the growth of eyelashes when applied in the FDA approved manner. It has now been found that bimatoprost may be used for the prevention or conversion of gray hair of humans or other mammals to the original color of such hair.

It is, therefore, a principal object of the present invention to provide a novel and effective treatment for the prevention of gray hair in mammals, especially humans.

It is, therefore, a principal object of the present inventions to provide a novel and effective treatment for the conversion of gray hair of humans or other mammals to the original pigment of the hair follicles.

Another object of the invention is to provide a method of stimulating hair pigmentation in humans and non-human animals that is compatible with various types of therapeutic agents or carriers and, therefore, would appear to be combinable with those which, by themselves, demonstrate some therapeutic activity, for prevention or converting gray hair to its original color.

Still another objective is the provision of a treatment for the conversion or prevention of gray hair which while effective for its intended purpose, is apparently non-toxic and relatively free of unwanted side effects.

An additional object of the invention herein disclosed and claimed is to provide a method for treating the graying of hair in men or women which can be applied by the patient under medical supervision no more stringent than that demanded for other topically-administered therapeutic agents.

Other objects of the invention are to provide a treatment for graying of hair in men or women which is safe, simple, painless, cosmetic in the sense of being invisible and easy to apply when compared with dying hair and the like.

### SUMMARY OF THE INVENTION

Bimatoprost and other cyclopentane(ene) heptan(en)oic acid amides are useful in the conversion of hair color to its original pigment or to generally darken hair.

This invention provides pharmaceutical compositions for topical application to enhance hair pigmentation comprising an effective amount of a cyclopentane(ene) heptan(en)oic acid, 2-cycloalkyl or arylalkyl compound represented by the formula I 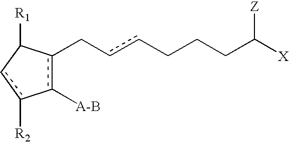
wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 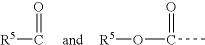
R⁵—C— and R⁵'O—C— wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above in free form or a pharmaceutically acceptable salt thereof, in association with a pharmaceutical carrier.

Preferably, the compound is a cyclopentane heptanoic acid, 2-(phenyl alkyl or phenyloxyalkyl) represented by the formula II 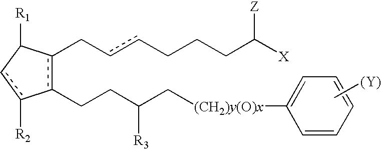
wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein said alkyl radical comprises from one to six carbon atoms, etc. n is 0 or an integer of from 1 to 3 and R₃ is ═O, —OH or —O(CO)R₆ wherein R₆ is as defined above or a pharmaceutically acceptable salt thereof.

More preferably the compound is a compound of formula III. 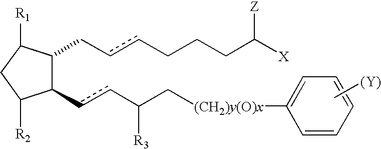
wherein hatched lines indicate a configuration, solid triangles are used to indicate β configuration.

More preferably y is 1 and x is 0 and R₁, R₂ and R₃ are hydroxy.

Most preferably the compound is cyclopentane N-ethyl heptanamide-5-cis-2-(3α-hydroxy-5-phenyl-1-trans-pentenyl)-3,5-dihydroxy, [1_{α},2_{β},3_{α},5_{α}], also known as bimatoprost.

Bimatoprost is an agent to promote the conversion of gray hair to the original pigment in hair follicles. Bimatoprost may be administered with suitable pharmaceutical carriers and can be in solid or liquid dosage form such as tablets, capsules, powders, soft gels, solutions, suspensions, emulsions, creams or ointments. A further object of this invention is to supply the compounds of this invention in a controlled-release formulation.

Bimatoprost can be administered orally, parenterally, for example, subcutaneously, intravenously, intramuscularly, intraperitoneally, by intranasal instillation or by application to mucous membranes via an aerosol spray or by application to the scalp or skin by ointment or a cream. Bimatoprost may be also applied topically to the skin, e.g. as a solution, suspension, emulsion, gel or aerosol spray.

The quantity of bimatoprost administered will vary depending on the patient and the mode of administration and can be any effective amount. The quantity of bimatoprost administered may vary over a wide range to provide in a unit dosage an effective amount from about 0.001 to 20 mg/kg of body weight of the patient per day to achieve the desired effect. For example, the desired affect can be obtained by consumption of a unit dosage form such as a tablet containing 1-200 mg of a cyclopentane(ene) heptan(en)oic acid amide compound of this invention taken 1-3 times daily.

These and other aspects of the invention will become apparent from the description of the invention which follows below.

### DETAILED DESCRIPTION OF THE INVENTION

Some examples of representative compounds useful in the practice of the present invention include the compounds shown in Table 1:

TABLE 1
cyclopentane heptenamide-5-cis-2-(3α-hydroxy-5-phenyl-1-trans-pent-
enyl)-3,5-dihydroxy, [1α, 2β, 3α, 5₆α]
cyclopentane N,N-dimethylheptenamide-5-cis-2-(3α-hydroxy-5-phenyl-
1-trans-pentenyl)-3,5-dihydroxy, [1α, 2β, 3α, 5α]
cyclopentane heptenylamide-5-cis-2-(3α-hydroxy-4-meta-chlorophen-
oxy-1-trans-pentenyl)-3,5-dihydroxy, [1α, 2_{β}, 3_{α}, 5_{α}]
cyclopentane heptenylamide-5-cis-2-(3α-hydroxy-4-trifluoromethylphen-
oxy-1-trans-pentenyl)-3,5-dihydroxy, [1α, 2β, 3α, 5α]
cyclopentane N-isopropyl heptenamide-5-cis-2-(3α-hydroxy-5-phenyl-1-
trans-pentenyl)-3,5-dihydroxy, [1α, 2β, 3α, 5α]
cyclopentane N-ethyl heptenamide-5-cis-2-(3α-hydroxy-5-phenyl-1-trans-
pentenyl)-3,5-dihydroxy, [1α, 2β, 3α, 5α]
cyclopentane N-methyl heptenamide-5-cis-2-(3α-hydroxy-5-phenyl-1-
trans-pentenyl)-3,5-dihydroxy, [1α, 2β, 3α, 5α]
cyclopentane heptenamide-5-cis-2-(3α-hydroxy-4-meta-chlorophenoxy-
1-trans-butenyl)-3,5-dihydroxy, [1α, 2β, 3α, 5α]

One presently preferred compound for use in the practice of the present invention is cyclopentane N-ethyl heptanamide-5-cis-2-(3α-hydroxy-5-phenyl-1-trans-pentenyl)-3,5-dihydroxy, [1(1_{α},2_{β},3_{α},5_{α}], also known as bimatoprost and sold under the name of Lumigan® by Allergan, Inc., California, USA. This compound has the following structure:

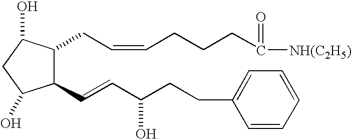

The synthesis of the compounds described above has been disclosed in U.S. Pat. No. 5,607,978. This patent also shows, particularly in Examples 1, 2, 5 and 7 that these compounds are not prostaglandins, in that they do not behave as prostaglandins in art-recognized assays for prostaglandin activity. The invention thus relates to the use of the above compounds, or prodrugs of the active compounds, for prevention or conversion of gray hair in mammals, e.g. humans. As used herein, gray hair includes hair associated with the scalp, eyebrows, eyelids, beard, and other areas of the skin of animals, e.g. humans.

In accordance with one aspect of the invention, the compound is mixed with a dermatologically compatible vehicle or carrier. The vehicle which may be employed for preparing compositions of this invention may comprise, for example, aqueous solutions such as e.g., physiological salines, oil solutions or ointments. The vehicle furthermore may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid; these may be used for increasing the viscosity. Furthermore, it is also possible to use soluble or insoluble drug inserts when the drug is to be administered.

The invention is also related to dermatological compositions for topical treatment for the conversion or prevention of gray hair which comprise an effective amount of one or more compounds as defined above and a dermatologically compatible carrier. Effective amounts of the active compounds may be determined by one of ordinary skill in the art but will vary depending on the compound employed, frequency of application and desired result, and the compound will generally range from about 0.0000001 to about 50%, by weight, of the dermatological composition, preferably. from about 0.001 to about 50%, by weight, of total dermatological composition, more preferably from about 0.1 to about 30%, by weight of the composition.

The present invention finds application in all mammalian species, including both humans and animals. In humans, the compounds of the subject invention can be applied for example, to the scalp, face, beard, head, pubic area, upper lip, eyebrows, eyelids and other skin surfaces of the body. In animals raised for their pelts, e.g., mink, the compounds can be applied over the entire surface of the body to improve the overall pelt for commercial reasons. The process can also be used for cosmetic reasons in animals, e.g., applied to the skin of dogs and cats having gray patches due to aging or diseases causing graying.

The pharmaceutical compositions contemplated by this invention include pharmaceutical compositions suited for topical and local action.

The term “topical” as employed herein relates to the use of a compound, as described herein, incorporated in a suitable pharmaceutical carrier, and applied at the site of graying hair for exertion of local action. Accordingly, such topical compositions include those pharmaceutical forms in which the compound is applied externally by direct contact with the skin surface to be treated. Conventional pharmaceutical forms for this purpose include ointments, liniments, creams, shampoos, lotions, pastes, jellies, sprays, aerosols, and the like, and may be applied in patches or impregnated dressings depending on the part of the body to be treated. The term “ointment” embraces formulations (including creams) having oleaginous, water-soluble and emulsion-type bases, e.g., petrolatum, lanolin, polyethylene glycols, as well as mixtures of these.

Typically, the compounds are applied repeatedly for a sustained period of time topically on the part of the body to be treated, for example, the eyelids, eyebrows, skin or scalp. The preferred dosage regimen will generally involve regular, such as daily, administration for a period of treatment of at least one month, more preferably at least three months, and most preferably at least six months.

For topical use on the skin and the scalp, the compound can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Various matrices for slow release delivery may also be used. Typically, the dose to be applied on the scalp is in the range of about 0.1 ng to about 100 mg per day, more preferably about 1 ng to about 10 mg per day, and most preferably about 10 ng to about 1 mg per day depending on the compound and the formulation. To achieve the daily amount of medication depending on the formulation, the compound may be administered once or several times daily with or without antioxidants.

The invention is further illustrated by the following non-limiting examples:

### EXAMPLE 1

A male patient (age 72) began taking bimatoprost as a tablet. After taking the medicine (oral dosage: 2.times. 100 mg per dose, 3 doses per day) for four months, it was discovered that a significant portion of his gray hairs had gradually turned into the original pigment or dark brown color.

### EXAMPLE 2

A healthy male subject (age 61) with normal blood pressure volunteers to take the medicine for observation on hair growth promoting activity at the same dose reported in Example 1. Before taking the medicine he has few hairs left in the frontoparietal area of the head. Three months after taking the medicine, he begins to note an increase of hair density in the affected area. During the three to six months of the testing period, he further notes that he has to increase the hair cut frequency to once a month from once every two months. The newly grown hairs in the affected area are mostly dark brown. The response to the medicine is more sensitive in areas with most recent hair loss. The overall increase of the hair density in the affected area is very significant. The observation is terminated at the end of six months. During the observation period, the subject does not experience any untoward effects.

### EXAMPLE 3

A healthy male (age 47) with normal blood pressure volunteers to take the medicine for the same observation as in Example 2 at the dose described above. His hair condition is normal with no baldness. During the six month testing period, he collects in intervals the hair samples from regular daily combing. Before the testing and during the first month of testing, the hair samples collected have two types, namely completely dark brown or completely gray. At around 50 days into the testing period, a new type of hair, partly dark brown and partly gray, begins to appear in the hair samples; they represent about 3% of the gray hairs. In this subject, the gray hairs represent about 25% of his total hairs in the samples collected. It is noted that the dark brown part of the new type of hair is always associated with the lower part of the hair shaft. This is easily identifiable because the hair follicles are distinguishable at one end of hair shafts. The quality and thickness of the new type of hair is very similar to the other types of hairs of this subject. The ratio of length of the dark brown part to that of the gray part of the new type of hair varies from 1:5 to 4:1 in the samples collected during the five-month period. This varying ratio may reflect the stages of the growth cycle of each hair follicle examined. The observation was discontinued at the end of five months. No untoward effects were reported.

### EXAMPLE 4

Topical Cream

A topical cream is prepared as follows: Tegacid and spermaceti are melted together at a temperature of 70-80° C. Methylparaben is dissolved in about 500 gm of water and propylene glycol, polysorbate 80, and bimatoprost are added in turn, maintaining a temperature of 75-80° C. The methylparaben mixture is added slowly to the Tegacid and spermaceti melt, with constant stirring. The addition is continued for at least 30 minutes with additional stirring until the temperature has dropped to 40-45° C. Finally, sufficient water is added to bring the final weight to 1000 gm and the preparation stirred to maintain homogeneity until cooled and congealed.

### EXAMPLE 5

Topical Cream

A topical cream is prepared as follows: Tegacid and spermaceti are melted together at a temperature of 70-80° C. Methylparaben is dissolved in water and propylene glycol, polysorbate 80, and bimatoprost are added in turn, maintaining a temperature of 75-80° C. The methylparaben mixture is added slowly to the Tegacid and spermaceti melt, with constant stirring. The addition is continued for at least 30 minutes with additional stirring until the temperature has dropped to 40-45° C. Finally, sufficient water is added to bring the final weight to 1000 gm and the preparation stirred to maintain homogeneity until cooled and congealed.

The composition is applied to human scalp once daily to convert gray hair to its original color.

### EXAMPLE 6

Topical Ointment

An ointment containing 2% by weight bimatoprost is prepared as follows:

White petrolatum and wool fat are melted, strained and liquid petrolatum is added thereto. The bimatoprost, zinc oxide, and calamine are added to the remaining liquid petrolatum and the mixture milled until the powders are finely divided and uniformly dispersed. The mixture is stirred into the white petrolatum, melted and cooled with stirring until the ointment congeals.

The foregoing ointment can be applied topically to mammalian skin for converting gray hair to its original color, and can be prepared by omitting the zinc oxide and calamine.

### EXAMPLE 7

Ointment

A dermatological ophthalmic ointment containing 10% by weight bimatoprost is prepared by adding the active compound to light liquid petrolatum. White petrolatum is melted together with wool fat, strained, and the temperature adjusted to 45-50° C. The liquid petrolatum slurry is added and the ointment stirred until congealed. Suitably the ointment is packaged in 30 gm tubes.

The foregoing ointment can be applied to the scalp to prevent the graying of hair.

### EXAMPLE 8

Solution

An aqueous solution containing 5%, by weight, bimatoprost is prepared as follows. Bimatoprost is dissolved in water and the resulting solution is sterilized by filtration. The solution is aseptically filled into sterile containers.

The composition so prepared can be used in the topical treatment of gray hair by application to the scalp daily.

### EXAMPLE 9

Lotion

A sample of bimatoprost is dissolved in the vehicle of N-methyl pyrrolidone and propylene glycol. The composition can be used for application to dogs or cats to treat gray hair.

### EXAMPLE 10

Aerosol

An aerosol containing approximately 0.1% by weight bimatoprost is prepared by dissolving the bimatoprost in absolute alcohol. The resulting solution filtered to remove particles and lint. This solution is chilled to about minus 30° C. To the solution is added a chilled mixture of dichlorodifluoromethane and dichlorotetrafluoroethane.

Thirteen ml plastic-coated amber bottles are cold filled with 11.5 gm each of the resulting solution and capped.

The composition can be sprayed on the scalp daily to prevent the graying of hair.

### EXAMPLE 11

Dusting Powder

A powder of the compound bimatoprost is prepared by mixing in dry form with talcum powder at a weight/weight ratio of 1:10. The powdered mixture is dusted on the fur of minks or other commercially valuable fur bearing animals and show animals for converting gray hair to its original pigment color.

### EXAMPLE 12

Related Compounds

Following the procedure of the preceding Examples, compositions are similarly prepared substituting an equimolar amount of a compound of Table 1 for the bimatoprost disclosed in the preceding Examples. Similar results are obtained.

The present invention is not to be limited in scope by the exemplified embodiments, which are intended as illustrations of individual aspects of the invention. Indeed, various modifications for the invention in addition to those shown and described herein will be come apparent to those skilled in the art from the foregoing description. Such modifications are intended to fall within the scope of the appended claims.

1. A method of preventing the graying of mammalian hair or converting gray hair to the original pigment in hair follicles of a mammal in need thereof which comprises administering to said mammal a pharmaceutical composition comprising an effective amount of a compound represented by formula I 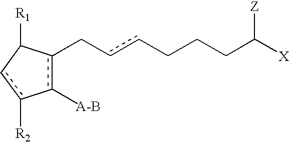
wherein the dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 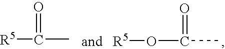
R⁵—C— and R⁵—O—C—, wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)mR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

2. The method of claim 1 wherein the concentration of the compound applied is from about 0.0000001% to about 50% by weight of the composition.

3. The method of claim 1 wherein the compound is a compound of formula (III). 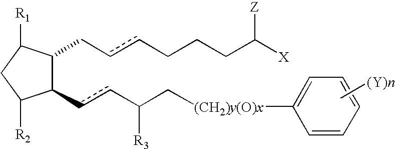
wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

4. The method of claim 3 wherein the compound is bimatoprost or a pharmaceutically acceptable salt thereof

5. The method of claim 1 wherein the compound of formula I is applied systemically.

6. The method of claim 1 wherein the compound of formula I is applied topically.

7. The method of claim 1 wherein the compound of formula I is applied by aerosol.

8. The method of claim 1 wherein the compound of formula I is applied as a cream.

9. The method of claim 1 wherein the compound of formula I is applied as a lotion.

10. The method of claim 1 wherein the compound of formula I is applied as a solution.
